(19) United States
(12) Patent Application Publication
(10) Pub. No.: US 2006/0088815 A1
(43) Pub. Date: Apr. 27, 2006

(54) Method for evaluation of endocrine disruptive action

(75) Inventors: Suguru Kawato, Tokyo (JP); Norio Takata, Tokyo (JP); Mari Ikeda, Tokyo (JP)

Correspondence Address: OLIFF & BERRIDGE, PLC, P.O. BOX 19928, ALEXANDRIA, VA, 22320, US

(73) Assignee: THE UNIVERSITY OF TOKYO, TOKYO (JP)

(21) Appl. No.: 11/208,859

(22) Filed: Aug. 23, 2005

(30) Foreign Application Priority Data
Oct. 22, 2004 (JP) 2004-308264

(51) Int. Cl. C12Q 1/00

(52) U.S. Cl. 435/4

(57) ABSTRACT

There is provided a method for a quick and easy evaluation of a substance which is an object of evaluation for its endocrine disruptive action. An evaluating method according to the present invention is a method for the evaluation of endocrine disruptive action which is characterized in comprising a step where the object substance is brought into contact with a cell group in which an electrode is arranged, a step where an agonist to a hormone receptor is brought into contact as a comparative substance with a cell group in which an electrode is arranged, and a measuring step where electrophysiological responses in the cell groups are measured via the electrode before and after contacting the object substance and before and after contacting the comparative substance, respectively.

### BACKGROUND OF THE INVENTION

The present invention relates to a method for specifying a substance having an endocrine disruptive action and, more particularly, it relates to a method for a quick and simple evaluation of endocrine disruptive action.

As one mechanism for adjustment of generation, growth, metabolism, etc. of a living body, an endocrine system by hormones (growth hormone, sex hormone, etc.) secreted into blood from specific cells among cells constituting the living organisms has been known.

An example of the endocrine system is a mechanism in which female sex hormone (estrogen) secreted by follicle cells of the ovary is circulated in blood and bonded to an estrogen receptor in other cells whereby a function which is specific to estrogen is expressed in the other cells.

With regard to the endocrine system as such, there are some cases where an extrinsic substance which induces the same action as a hormone by bonding to a hormone receptor in spite of the fact that it is a substance being different from the hormone which is an inherent agonist to the hormone receptor becomes a problem as a so-called endocrine disrupter.

It has been reported that, for example, diethylstilbestrol (DES), which is known as the so-called synthetic estrogen, has been much used with an object of prevention of miscarriage and, as a result, it generates tardive cancer, etc. to exposed genital organs of females during a prenatal period and DES has been known as one endocrine disruptor.

With regard to the endocrine disruptor as such, although there are substances such as DES where the endocrine disruptive action has been confirmed, it is though that there are still unknown substances where no endocrine disruptive action has been confirmed yet.

Accordingly, in order to prevent the bad effects of known or unknown substances on human health or ecosystems of the natural world, it is necessary to correctly evaluate the endocrine disruptive action of substances and to specify the endocrine disrupter.

Under such circumstances, in order to specify an endocrine disrupter, there have been attempts such as a method where, when a candidate substance is applied to a parent generation of an animal for example, influence of the candidate substance on a generation process of the child generation thereof is evaluated, and a method where influence of a candidate substance on the growth ability of incubated cells is evaluated (Non-Patent Document 1, etc.).

[Non-Patent Document 1] Newbold, R. R., et al., Carcinogenesis, 19, 1655-1663, 1998.

However, in the above-mentioned conventional evaluation methods, a long-term action observed after a relatively long time via a hormone receptor existing in nuclei of cells as an acting route of the hormone is used as an index, and therefore, a long time is needed to obtain the result, which is less convenient when evaluation is conducted for many candidate substances.

It has been further reported that, with regard to 67 kinds of substances which have been listed as candidates for an endocrine disrupter by the Ministry of the Environment of Japan until now, no apparent endocrine disruptive action for mammals has been confirmed. In 2004, it was announced that endocrine disruptors will be specified again from about one thousand kinds of chemical substances where toxicity is doubtful.

For such a purpose, there has been a demand for a novel method for the evaluation of endocrine disruptive action as one of the methods for specifying endocrine disruptors.

The present invention has been achieved in view of the above-mentioned problem, and one of the objects of the present invention is to provide a method by which endocrine disruptive action of the substances to be evaluated can be quickly and easily evaluated.

### SUMMARY OF THE INVENTION

In order to solve the above-mentioned problems that have existed up to now, a method for evaluation of endocrine disruptive action according to one of the embodiments of the present invention is a method for the evaluation of endocrine disruptive action of an object substance which is characterized in comprising a step where the object substance is brought into contact with a cell group in which an electrode is arranged, a step where an agonist to a hormone receptor is brought into contact, as a comparative substance, with a cell group in which an electrode is arranged and a measuring step where electrophysiological responses in the cell groups are measured via the electrode before and after bringing the object substance into contact therewith and before and after bringing the comparative substance into contact therewith, respectively.

Further, a method for evaluation of endocrine disruptive action according to another embodiment of the present invention is a method for the evaluation of endocrine disruptive action of an object substance which is characterized in comprising a step where the object substance is brought into contact with a cell group in which an electrode is arranged, a step where an antagonist to a hormone receptor is brought into contact, as a comparative substance, with a cell group in which an electrode is arranged followed by bringing the object substance into contact therewith and a measuring step where electrophysiological responses in the cell groups are measured via the electrodes before and after bringing the object substance into contact therewith.

In accordance with the present invention, it is now possible to provide a method where endocrine disruptive action of a substance which is an object for the evaluation can be quickly and easily evaluated.

### BRIEF DESCRIPTION OF THE DRAWINGS

FIG. 1 is a graph which shows an example of results of measurement of excitatory postsynaptic potential in the evaluating method for endocrine disruptive action in an embodiment of the present invention.

FIG. 2 is a graph which shows an example of results of measurement of influence of estradiol on excitatory postsynaptic potential in the evaluating method for endocrine disruptive action in an embodiment of the present invention.

FIG. 3 is a graph which shows an example of results of measurement of influence of DES on excitatory postsynaptic potential in the evaluating method for endocrine disruptive action in an embodiment of the present invention.

### DESCRIPTION OF THE PREFERRED EMBODIMENTS

As described in the following, a method for evaluation of endocrine disruptive action according to the present invention will be illustrated by referring to the drawings. Incidentally, the method for evaluation of endocrine disruptive action according to the present invention is not limited to the following embodiments only.

Firstly, outline of the method for evaluation of endocrine disruptive action according to the first embodiment of the present invention (hereinafter, referred to as the present evaluation method 1) will be illustrated. In the present evaluation method 1, attention is paid to an acute action induced within a very short time discovered by the present inventor among the hormone actions induced by a hormone receptor owned by cells of a living body.

Thus, the present inventor has found, as a result of intensive studies, that when a substance which has been already known as a so-called environmental hormone (such as DES) for example is brought into contact with cells having a hormone receptor, an electrophysiological response (such as changes in cell membrane potential) which is the same as that in a hormone which is an inherent agonist to the hormone receptor is induced in the cell within a very short time after the contact. Incidentally, it has been thought that the hormone receptor according to the acute hormone-like action is mainly present on a cell membrane.

On the basis of the original finding by the present inventor as mentioned above, the present evaluation method 1 is to evaluate whether a substance which is an object for evaluation of endocrine disruptive action induces the same electrophysiological response to the cell group having a hormone receptor as the agonist to the hormone receptor, and to specify the object substance inducing the hormone-like electrophysiological response as a candidate for an endocrine disrupter.

Here, with regard to a cell group for measuring the electrophysiological response in the present evaluation method 1, anything may be used regardless of the type of animal and tissue it is derived from provided that at least some of the cells included in the cell group have a hormone receptor (receptor to, for example, steroid hormone (including stress hormone, male sex hormone, female sex hormone, etc.)) and that, when an agonist is bonded to the hormone receptor, an electrophysiological response results. Particularly, that derived from brain tissue and nerve tissue is preferred since it shows a significant electrophysiological response.

To be specific, tissue slices which are thinly cut out from tissues of a living body using, for example, a vibratome may be used as the cell group. In that case, slices of brain tissue, slices of nerve tissue, etc., for example, may be used as a cell group. Brain tissue slices such as the hippocampal region of a rat, mouse, etc. may be used particularly advantageously. When brain tissue slices of the hippocampal region are used, slices transversely cut along a long axis are advantageous. Here, the slice transversely cut along a long axis means a slice which is cut vertically to a long axis of the hippocampus. With regard to the tissue slices, those where thickness is within a range of about 150 to 400 μm may be preferably used and, particularly, those where the thickness is about 300 μm may be used advantageously. The reason for this is that when thickness of the tissue slice is smaller than about 150 μm, most of the nerve cells contained in the tissue slice are severely damaged in the cell body or dendrite upon preparation of the slice, which makes the slice hard to use as a healthy tissue, while when the thickness is larger than about 400 μm, supply of oxygen from a solution in which the tissue slice is soaked to nerve cells inside the tissue slice is insufficient, which also makes it difficult to use the slice as a healthy tissue. With regard to the cell group, it is also possible to use primary cells isolated from living body tissue by a known cell preparation method using a digestive enzyme or the like, established cell line, cells subjected to a genetic recombination treatment, etc. In that case, incubated primary nerve cells isolated from brain tissue or nerve tissue, incubated established nerve cell line, incubated nerve cells genetically recombined, etc. may be preferably used as a cell group.

In the present evaluation method 1, an electrode is arranged in the cell group for measurement of its electrophysiological response and used. The electrode may be anything as long as it is able to measure an electrophysiological response such as changes in cell membrane potential of a cell group contacting the electrode. For example, it is possible to use a glass electrode which pierces the cells or a multiple-electrode probe where plural electrodes are regularly arranged on a substrate. In view of the fact that plural cells can be measured easily and stably, the multiple-electrode probe can be used advantageously.

With regard to the arrangement of the electrode in the cell group, it may be such that plural electrodes are arranged to contact at least a part of the cell group existing near the surface of the tissue slice (a cross section cut out from the tissue) when tissue slices are used as a cell group. In that case, it is possible to arrange the electrode by, for example, placing the tissue slices on plural electrodes of the multiple-electrode probe. When, for example, hippocampal tissue slices of rat, mouse, etc. are used as a cell group, it is possible to arrange in such a manner that each of pyramidal cells existing in the hippocampal cornu ammonis and granular cells existing in the hippocampal dentate gyrus are broght into contact with the electrodes. When a cultured cell group is used as a cell group for example, arrangement of the electrode may also be carried out in such a manner that the plural electrodes are brought into contact with at least some of the cells contained in the incubated cell group. In that case, it is also possible to arrange the electrodes by adhering the incubated cell group onto the plural electrodes of the multiple-electrode probe.

Now, the present evaluation method 1 will be more specifically illustrated. The present evaluation method 1 includes a step for contacting the object substance, a step for contacting the comparative substance and a step for measurement. Hereinafter, each of those steps will be illustrated in that order. Incidentally, in the present evaluation method 1, illustration will be given taking the case where two cell groups of the first cell group and the second cell group are used as an example. Thus, the first cell group is used in the step for contacting the object substance and the step for measurement therefor while the second cell group is used in the step for contacting the comparative substance and the step for measurement therefor.

With regard to the first cell group and the second cell group, those having the same hormone receptor are used. For example, when a brain tissue slice cut out from one brain tissue is used as a cell group, it is possible that, among plural brain tissue slices prepared, one sheet of the tissue slices is used as the first cell group and another sheet of the tissue slices is used as the second group. When an incubated cell group is used as the cell group, it is possible that, among the cultured cell group where incubating conditions such as subcultured numbers are the same, a part of the cell group is used as the first cell group while another part of the cell group is as the second cell group. As described in the following, the first cell group and the second cell group may be simply referred to as a cell group when there is no particular necessity to differentiate them.

In a step of contacting an object substance, the object substance is brought into contact with a first group in which an electrode is arranged. With regard to the object substance, there is no particular limitation as long as it is a substance which is an object for evaluating whether it has an endocrine disruptive action. Thus it is possible to use, for example, a substance which has already been reported to have any hormone-like action, a substance where its chemical structure is known and which is suspected to have an endocrine disruptive action from the chemical structure, and a substance where chemical structure and action are unknown.

To be more specific, in this step for contacting the object substance, the first cell group in which an electrode is arranged is soaked in an aqueous solution containing the object substance whereupon the object substance is brought into contact with the first cell group. Thus, for example, an aqueous solution containing no object substance is filled in a predetermined container, the first cell group is soaked in the aqueous solution for a predetermined period and the aqueous solution in the container is exchanged with an aqueous solution containing an object substance, whereby the first cell group is brought into contact with the object substance. Incidentally, the aqueous solution containing the object substance may be perfused to a vessel containing the first cell group, and a concentration of the object substance to be added to the aqueous solution may be freely adjusted.

With regard to the aqueous solution used here, that where compositions, concentrations, etc. of salts (such as sodium and potassium) and nutritive components (such as glucose and amino acids) are freely adjusted may be used so that the living state of the first cell group can be maintained depending upon the type (tissue from which the cell group is derived, type of cells), etc. of the first cell group. The aqueous solution is also used in the succeeding steps.

In a step of contacting a comparative substance, an agonist to a hormone receptor in the second group is contacted as a comparative substance to the second cell group in which electrode is arranged. With regard to the agonist, an inherent agonist to the hormone receptor in at least a part of the cells contained in the cell group may be advantageously used. To be more specific, when a cell group containing cells having a steroid hormone receptor such as an estrogen receptor is used, a steroid hormone such as commercially available estrogen may be used by dissolving in an aqueous solution in any concentration. With regard to the agonist, it is also possible to use stress steroid such as corticosterone, male sex hormone such as testosterone and 5-dihydrotestosterone, female sex hormone such as estradiol and estron, etc.

To be more specific, in this step for contacting a comparative substance, the second cell group where an electrode is arranged is soaked in an aqueous solution containing an agonist so that the agonist is brought into contact with the second cell group. Thus, for example, an aqueous solution containing no agonist is filled in a predetermined container, the second cell group is soaked in the aqueous solution for a predetermined period and the aqueous solution in the container is exchanged with an aqueous solution containing an agonist so that the second cell group is brought into contact with the agonist. In that case, the aqueous solution to which the agonist is added may be perfused into a container in which the second cell group is placed. It is also possible for the concentration of the agonist to be added to the aqueous solution to be freely adjusted.

In a measuring step, electrophysiological responses of the first cell group before and after contacting an object substance in the above step for contacting an object substance, and electrophysiological responses of the second cell group before and after contacting a comparative substance in the above step for contacting a comparative substance, are measured via electrodes which are arranged in the cell groups.

To be more specific, in this measuring step, a predetermined degree of stimulation is applied to at least some cells contained in the cell groups before and after contacting an object substance to the first cell group and before and after contacting an agonist to the second cell group for example, and the electrophysiological responses induced by the stimulation are measured.

Here, with regard to the stimulating method in this measuring step, there is no particular limitation therefor as long as an electrophysiological response of the cells is induced by the stimulation and, when brain tissue slices or incubated nerve cells are used as a cell group, examples thereof are electrical stimulation of presynaptic fiber projected from nerve cells contained in the cell group, and local spraying of a neurotransmitter to the nerve cells using a glass pipette in which the electrical stimulation is preferred since it can be conducted easily and stably. When, for example, a muscular tissue is used as a cell group, the same stimulating method can also be used for nerve cells forming synapse in the muscular tissue. When, for example, a neurotransmitter receptor which is able to change its property by bonding of agonist to a hormone receptor and an established cell transfected with gene of the hormone receptor are used as a cell group, it is also possible to use a stimulating method in which the established cell is brought into contact with the neurotransmitter.

When electrical stimulation is used as a stimulating method, an example is that electrical stimulation of a predetermined strength is applied from some electrodes, among plural electrodes arranged in a cell group, at a predetermined time interval to the cells contacting the electrodes and, at the same time, changes, etc. in cell membrane potential induced by the electrical stimulation from other electrodes can be repeatedly measured. To be more specific in that case, it is possible to use a method where accumulated potential is measured using a glass electrode, a method where intracellular potential is measured from a single cell by a glass electrode piercing method, a method where intracellular potential is measured from a single cell by a patch clump method using a glass electrode, a method using a multiple-electrode probe, etc. and, among these methods, a method where accumulated potential of a cell group contacting plural electrodes of a multiple-electrode probe is measured is particularly advantageously used as a simple and reliable method.

Thus, in a measuring step when an electrical stimulation method using a multiple-electrode probe is used, an example is that brain tissue slices of a hippocampal region are used as a cell group, plural electrodes of the multiple-electrode probe are arranged in the brain tissue slices, estrogen is used as an agonist, excitatory postsynaptic potential of the first brain tissue slices soaked in an aqueous solution containing no object substance is first measured via the electrodes, then the first brain tissue slices are soaked in an aqueous solution containing an object substance and its excitatory postsynaptic potential is further measured. With regard to the second brain tissue slices, an excitatory postsynaptic potential of the second brain tissue slices soaked in an aqueous solution containing no estrogen is first measured, then the second brain tissue slices are soaked in an aqueous solution containing estrogen and an excitatory postsynaptic potential is further measured. Incidentally, the aqueous solution containing the object substance or estrogen may be perfused in a container in which the brain tissue slices are placed.

In that case, when the value of excitatory postsynaptic potential before and after contacting of the first brain tissue slices with an object substance and the value of excitatory postsynaptic potential before and after contacting of the second brain tissue slices with estrogen have a similar measured result in the measuring step, it is confirmed that the object substance has a high possibility of having the same hormone-like action as estrogen. Thus, it is confirmed that the object substance has a high possibility of being an endocrine disrupter causing an endocrine disruptive action via an estrogen receptor owned by at least some cells contained in the brain tissue slices. On the other hand, when the excitatory postsynaptic potential before and after contacting of the first brain tissue slices with an object substance and the excitatory postsynaptic potential before and after contacting of the second brain tissue slices with estrogen are similar, the object substance is confirmed to have no endocrine disruptive action, at least via estrogen receptor.

As such, in accordance with the present evaluation method 1, an endocrine disruptive action by an object substance can be measured as an electrophysiological response of a cell group, and therefore, an endocrine disruptive action of the object substance can be measured very quickly and easily.

In addition to the above-mentioned step of contacting an object substance, step of contacting a comparative substance and step of measurement, the present evaluation method 1 may further contain a step of contacting a stimulant where a stimulant which causes a change in an electrophysiological response of a cell group is brought into contact with a cell group in contact with an object substance and a cell group in contact with a comparative substance.

With regard to the stimulant used here, anything may be used without particular limitation as long as it is a substance which causes a change to an electrophysiological response of a cell group by a route that is different from a route via a hormone receptor and it changes the degree of change in the electrophysiological response between the case where the cell group is previously brought into contact with an agonist and the case where the cell group is not brought into contact with an agonist. To be more specific, when a nerve cell group is used as a cell group for example, a substance which causes certain changes in excitatory postsynaptic potential of the nerve cells before and after contacting of a stimulant to the nerve cell group, and further causes changes in the degree of change in the excitatory postsynaptic potential between the case where the nerve cell and estrogen are previously contacted and the case where they are not contacted, may be used as a stimulant. In that case, N-methyl-D-aspartic acid (NMDA) or the like which causes so-called long-term depression of excitatory postsynaptic potential to a nerve cell group or the like contained in brain tissue slices may be used as a stimulant. Incidentally, in that case, the acting concentration (concentration of being dissolved in an aqueous solution) of the NMDA is preferred to be within a range of 10 to 50 μM while its acting time (time for soaking the nerve cell group in an aqueous solution to which NMDA is added) is preferred to be within 10 minutes, and the acting concentration of 30 μM and the acting time of 3 minutes are particularly advantageous. This is because when the acting concentration of NMDA is less than 10 μM, its stimulating action is insufficient and, when the acting concentration of NMDA is more than 50 μM or the acting time is longer than 10 minutes, the nerve cell group may be dead. Incidentally, it has been confirmed that, within the above-mentioned appropriate ranges of action concentration and acting time, a sufficient stimulating action is available where the nerve cells are kept in a healthy state. Besides the above, it is also possible that, when hippocampal tissue slices are used as a cell group for example, (RS)-3,5-dihydroxyphenylglycine (DHPG) or the like, which is an agonist to a type I metabolic type glutamic acid receptor causing a long-term depression to the nerve cell group contained the hippocampus brain tissue slices, is used.

To be more specific, in this step of contacting a stimulant, an example is that the first cell group is first soaked in an aqueous solution to which an object substance is added in a certain container while the second cell group is soaked in an aqueous solution to which an agonist is added and, after that, the aqueous solution in the container is exchanged with the aqueous solution to which the stimulant is added, whereby the cell group is brought into contact with the stimulant. Incidentally, in that case, it is also possible for the aqueous solution to which a stimulant is added to be perfused to a container in which the cell group is placed. Further, concentration of the stimulant to be added to the aqueous solution may be freely adjusted.

When the present evaluating method 1 includes the step for contacting the stimulant, in addition to the electrophysiological response of the first cell group before and after contacting the object substance, and the electrophysiological response of the second cell group before and after contacting the comparative substance, an electrophysiological response of the cell groups before and after contacting the stimulant is also measured in a measuring step.

To be more specific, in this measuring step, an electrophysiological response is first measured for the first cell group soaked in an aqueous solution containing no object substance, then the first cell group is soaked in an aqueous solution containing an object substance to measure an electrophysiological response and, further, the first cell group is soaked in an aqueous solution containing a stimulant to measure an electrophysiological response. Further, in this measuring step, an electrophysiological response is first measured for the second cell group soaked in an aqueous solution containing no agonist, then the second cell group is soaked in an aqueous solution containing an agonist to measure an electrophysiological response and, further, the second cell group is soaked in an aqueous solution containing a stimulant to measure an electrophysiological response. As such, in the present evaluating method 1, electrophysiological responses of cell groups may be continuously measured in terms of time throughout the whole step.

Thus, for example, when an excitatory postsynaptic potential of brain tissue slices is measured as an electrophysiological response using, for example, brain tissue slices as a cell group, estrogen is used as an agonist and NMDA is used as a stimulant. The first brain tissue slice and the second brain tissue slice are subjected to measurement of excitatory postsynaptic potential without contacting any of stimulant, estrogen and NMDA, the first brain tissue slice is then brought into contact with an object substance while the second brain tissue slice is brought into contact with estrogen to measure each excitatory postsynaptic potential in the first brain tissue slice and second brain tissue slice, and after that, the first brain tissue slice and second brain tissue slice are further brought into contact with NMDA, as a result of which an excitatory postsynaptic potential for each of the brain tissue slices is further measured.

When the degree of change in excitatory postsynaptic potential in the first brain tissue slice brought into contact with NMDA after contacting an object substance and the degree of change in excitatory postsynaptic potential in the second brain tissue slice brought into contact with NMDA after contacting estrogen are the same in the above measuring step, it is confirmed that the object substance has a high possibility of being an endocrine disrupter having an action of causing a change in excitatory postsynaptic potential of brain tissue slice via an estrogen receptor.

Therefore, in the present evaluating method 1 including a step of contacting a stimulant, a cell group is soaked in an aqueous solution containing a stimulant so that the stimulant is brought into contact with the whole cell group, and changes in an electrophysiological response in the cell group as a whole by the stimulant or the object substance can be measured reliably even in the case where, for example, cells on which the stimulant or the object substance acts are present exist in only in a part of the cell group. Further, in that case, an electrophysiological response of the cell group is significantly expressed by the stimulant, and therefore precision in the measurement of effect of an object substance or a comparative substance on the electrophysiological response can be improved. Consequently, in accordance with the present evaluating method 1, an endocrine disruptive action by an object substance can be easily and reliably evaluated as an electrophysiological response for the whole cell group.

Now, an evaluating method for an endocrine disruptive action in accordance with the second embodiment of the present invention (hereinafter, it will be referred to as the present evaluating method 2) will be illustrated. The present evaluating method 2 has focused on an acute action which is induced within a very short time in a hormone action caused via a hormone receptor owned by cells in a living body, the same as in the above-mentioned present evaluating method 1.

Thus, the present inventor has conducted intensive studies repeatedly, and as a result has found that when a substance which has been known as a so-called environmental hormone is brought into contact with cells having a hormone receptor, the same electrophysiological response as in the case of an agonist to the hormone receptor is induced in the cells within a very short time after the contact, while when the cell group is brought into contact with an antagonist to a hormone receptor and then brought into contact with the environmental hormone, the same electrophysiological response as in the agonist is not induced or, in other words, a hormone-like electrophysiological response via a hormone receptor is inhibited by the antagonist.

The present evaluating method 2 is to evaluate, on the basis of a unique finding by the present inventor, whether the object substance induces an electrophysiological response in cells having a hormone receptor, and also to evaluate whether the electrophysiological response is inhibited by an antagonist. Therefore, according to the present evaluating method 2, it is possible to specify an object substance which induces a hormone-like electrophysiological response being inhibited by an antagonist as a highly possible endocrine disruptor.

To be more specific, the present evaluating method 2 includes a step for contacting an object substance where an object substance is brought into contact with a cell group in which an electrode is arranged, a step for contacting a comparative substance where an antagonist to a hormone receptor is contacted as a comparative substance to a cell group in which an electrode is arranged and then an object substance is contacted thereto, and a measuring step where, with regard to the cell group, electrophysiological responses before and after contacting an object substance are measured via the electrode. In the present evaluating method 2, illustration will be given by taking a case using two cell groups comprising a first cell group and a second cell group as an example, the same as in the above-mentioned present evaluating method 1.

In a step of contacting an object substance in the present evaluating method 2, an object substance is brought into contact with the first cell group in which an electrode is arranged, the same as in a step of contacting an object substance in the above-mentioned present evaluating method 1. In a step of contacting a comparative substance in the present evaluating method 2, the second cell group in which an electrode is arranged is first soaked in an aqueous solution containing an antagonist to a hormone receptor so that the antagonist is brought into contact with the second cell group and then the second cell group is soaked in an aqueous solution containing an object substance so that the object substance is brought into contact with the second cell group.

With regard to the antagonist, any substance may be advantageously used as long as it bonds to a hormone receptor owned by at least some cells contained in the cell group but does not induce a hormone action which is induced by an inherent agonist to the hormone receptor. To be more specific, when a cell group having an estrogen receptor is used, an estrogen inhibitor such as commercially available 17α-estradiol can be used by dissolving in an aqueous solution in any concentration.

In a measuring step in the present evaluating method 2, the first cell group in which an electrode is arranged is first soaked in an aqueous solution containing no object substance to measure its electrophysiological response and then the first cell group is soaked in an aqueous solution containing an object substance to measure its electrophysiological response. Further, in this measuring step, the second cell group in which electrode is arranged is first soaked in an aqueous solution containing an antagonist to measure its electrophysiological response, and then the second cell group is soaked in an aqueous solution containing an object substance to measure its electrophysiological response.

To be more specific, when, for example, a brain tissue slice is used as a cell group and an estrogen inhibitor is used as an antagonist, changes in cell membrane potential before and after contacting an object substance in the first brain tissue slice brought into contact with an object substance without contacting an antagonist in this measuring step are not measured in the second brain tissue slice brought into contact with an object substance after contacting an antagonist, it is confirmed that there is a high possibility that the object substance induces the change in the cell membrane potential via an estrogen receptor or, in other words, it is an endocrine disruptor having a hormone-like action via an estrogen receptor.

As such, in the present evaluating method 2, an endocrine disruptive action by an object substance can be measured as an electrophysiological response and, therefore, an endocrine disruptive action of an object substance can be evaluated very quickly and easily.

The present evaluating method 2 may further contain a step of contacting a stimulant the same as in the above-mentioned evaluating method 1 in addition to the above-mentioned step of contacting an object substance, a step of contacting a comparative substance and a measuring step. In that case, when, for example, an excitatory postsynaptic potential of a brain tissue slice is measured as an electrophysiological response using a brain tissue slice as a cell group, using an estrogen inhibitor as an antagonist and using NMDA as a stimulant, an excitatory postsynaptic potential for the first brain tissue slice without contacting an estrogen inhibitor is first measured followed by measuring an excitatory postsynaptic potential for the second brain tissue slice contacting an estrogen inhibitor and, subsequently, the first brain tissue slice and the second brain tissue slice are brought into contact with the object substance to measure the excitatory postsynaptic potential. After that, the first brain tissue slice and the second brain tissue slice are further brought into contact with NMDA to measure the excitatory postsynaptic potential.

In a measuring step, when a change in an excitatory postsynaptic potential in the first brain tissue slice contacting NMDA after contacting an object substance is not measured in the second brain tissue slice contacting an estrogen inhibitor, then contacting an object substance and further contacting NMDA, it is confirmed that the object substance has a high possibility of being an endocrine disruptor which has an action of causing a change in hormone-like excitatory postsynaptic potential via an estrogen receptor.

As such, in the present evaluating method 2 including a step of contacting a stimulant, an endocrine disruptive action by an object substance can be easily and reliably evaluated as an electrophysiological response for the whole cell group, the same as in the case of the evaluating method 1 including a step of contacting a stimulant.

### EXAMPLES

Now, an example of a specific embodiment for the above-mentioned evaluating method 1 will be illustrated. First, the preparation of a cell group and a setting method of experimental conditions are explained below. With regard to the cell group, a tissue slice which was a transversely cut in a hippocampal region along a long-axis which was cut out from a brain tissue of an adult rat using a vibratome (DSK ZERO 1; manufactured by Dosaka EM K.K.) was used. Thickness of the hippocampal tissue slice was made 300 μm.

For the measurement of an electrophysiological response of this hippocampal tissue slice, a multiple-electrode measuring apparatus MED 64 System (Alpha Med) was used. With regard to an electrode arranged in the hippocampal tissue slice, a multiple-electrode probe MED-P530A (Alpha Med) attached to this multiple-electrode measuring apparatus was used. On a substrate of this multiple-electrode probe, plural electrodes are previously arranged regularly with a predetermined interval (300 μm).

One sheet of the hippocampal tissue slice cut out by a vibratome was allowed to stand in a chamber placed on a substrate of one multiple-electrode probe and, at the same time, an artificial cerebrospinal fluid sufficiently saturated with a mixed gas of oxygen and carbon dioxide (95% oxygen gas and 5% carbon dioxide gas) was filled in the chamber whereby the hippocampal tissue slice was soaked in the artificial cerebrospinal fluid followed by being allowed to stand at room temperature for 1 hour or longer. With regard to the artificial cerebrospinal fluid, an aqueous solution containing 124 mM of NaCl, 1.25 mM of NaH₂PO₄.2H₂O, 5 mM of KCl, 2 mM of MgSO₄.7H₂O, 2 mM of CaCl₂, 22 mM of NaHCO₃ and 10 mM of glutamine was used.

In this artificial cerebrospinal fluid, the hippocampal tissue slice was placed on the multiple-electrode probe so that the cells included in each of three regions comprising one dentate gyrus and two cornu ammonis regions near the surface of the hippocampal tissue slice were brought into contact with plural electrodes on the multiple-electrode probe. Thus, a stimulating electrode which applies electrical stimulation to presynaptic fiber projecting to the nerve cell group and a recording electrode which records excitatory postsynaptic potential induced by the electrical stimulation in the nerve cell group as accumulated potential are arranged for each of pyramidal cells of cornu ammonis CA1 field, pyramidal cells of cornu ammonis CA3 field and granular cells of dentate gyrus.

Electrical stimulation to a hippocampal tissue slice by a stimulating electrode was carried out by repeated application of electrical stimulation at intervals of 15 seconds in the order of a stimulating electrode arranged in a cornu ammonis CA1 field, a stimulating electrode arranged in a cornu ammonis CA3 field, a stimulating electrode arranges in a dentate gyrus and, again, a stimulating electrode arranged in a cornu ammonis CA1 field. The excitatory postsynaptic potential induced by the electrical stimulation was recorded for about 30 minutes and, after confirming that the accumulated potential was stabilized, strength of electrical stimulation applied from the stimulating electrode was set in each of the following steps. Thus, first, strength of electrical stimulation to be applied to each site (cornu ammonis CA1 field, cornu ammonis CA3 field and dentate gyrus) on the hippocampal tissue slice was gradually increased starting from weak strength and, at the same time, accumulated potential was measured. When the strength of the measured accumulated potential was saturated (or became constant), the strength of the saturated accumulated potential was defined as maximum (100%) and strength of electrical stimulation to be applied to the hippocampal tissue slice from the stimulating electrode was set so as to make the value 50% of the above maximum value.

In this Example, DES was used as an object substance, estradiol, which is one example of estrogen, was used as an agonist to hormone receptor and NMDA was used as a stimulant. In the following step, contact of the hippocampal tissue slice with DES, estradiol or NMDA was conducted by perfusion of an artificial cerebrospinal fluid containing the DES, estradiol or NMDA and being fully saturated with a mixed gas in a chamber at a flow rate of about 2 ml per minute.

Each of the steps in the present invention will now be illustrated. In this Example, a case using four sheets of hippocampal tissue slices of the first, second, third and fourth hippocampal tissue slices prepared from the same brain tissue will be illustrated. Each of those hippocampal tissue slices was arranged with a stimulating electrode and a recording electrode of the above-mentioned multiple-electrode probe. First, each hippocampal tissue slice being arranged with the electrodes is repeatedly subjected to electrical stimulation at the above-set electrical stimulation strength together with perfusion of an artificial cerebrospinal fluid which did not contain any of DES, estradiol and NMDA, and an excitatory postsynaptic potential thereof was continuously (with a lapse of time) recorded for 20 minutes (hereinafter, this step will be referred to as a preliminary step).

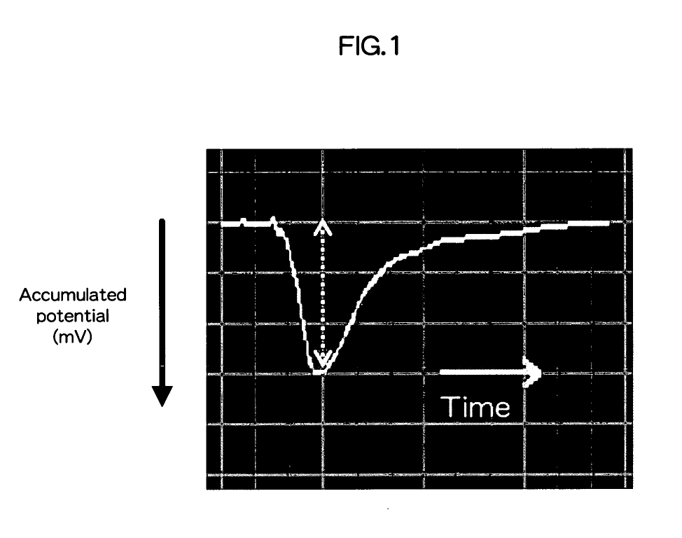

In this Example, result of the measurement of the excitatory postsynaptic potential was evaluated as follows. Thus, with regard to an excitatory postsynaptic potential, it is recorded as shown in FIG. 1, for example, in graph form where an abscissa represents time (minute(s)) while an ordinate represents accumulated potential (mV). Now, with regard to the measured result during 20 minutes in the above preliminary step, a mean value of absolute values (strength of the accumulated potentials) of heights of the wave of accumulated potential as shown by an arrow with a dotted line in FIG. 1 was calculated and, taking the mean value as 100%, it was evaluated what percenttage of the value of excitatory postsynaptic potential measured in the step, which will be mentioned later, corresponds to the mean value.

After the preliminary step, the perfusion liquid was exchanged with an artificial cerebrospinal fluid containing 1 nM DES in the case of the first hippocampal tissue slice, with an artificial cerebrospinal fluid containing 1 nM estradiol in the case of the second hippocampal tissue slice, with an artificial cerebrospinal fluid containing ethanol used as a solvent upon dissolving the above DES in the artificial cerebrospinal liquid in the case of the third hippocampal tissue slice, and with an artificial cerebrospinal fluid containing ethanol used as a solvent upon dissolving the above estradiol in the artificial cerebrospinal liquid in the case of the fourth hippocampal tissue slice, whereupon the excitatory postsynaptic potential for the electrical stimulation was continuously measured for 30 minutes more.

After the measurement of the excitatory postsynaptic potential during 30 minutes, NMDA was further added to the perfusion liquid to induce a long-term depression in each of the hippocampal tissues slices. Thus, the perfusion liquid was exchanged with an artificial cerebrospinal fluid containing 30 μM of NMDA and 1 nM of DES in the case of the first hippocampal tissue slice, with an artificial cerebrospinal fluid containing 30 μM of NMDA and 1 nM of estradiol in the case of the second hippocampal tissue slice, and with an artificial cerebrospinal fluid containing 30 μM of NMDA and ethanol in the cases of the third and fourth hippocampal tissue slices, whereupon excitatory postsynaptic potentials for the electrical stimulation were measured for 3 minutes more.

After that, the perfusion liquid was again exchanged with an aqueous solution containing no NMDA or, in other words, it was exchanged with an artificial cerebrospinal fluid containing 1 nM of DES in the case of the first hippocampal tissue slice, with an artificial cerebrospinal fluid containing 1 nM of estradiol in the case of the second hippocampal tissue slice, and with the above-mentioned ethanol-containing artificial cerebrospinal fluid in the cases of the third and fourth hippocampal tissue slices, whereupon excitatory postsynaptic potentials to the electrical stimulation were continuously measured for a further 60 minutes. Exchange of the perfusion liquid in each of the above steps was continuously conducted together with the measurement of the excitatory postsynaptic potential.

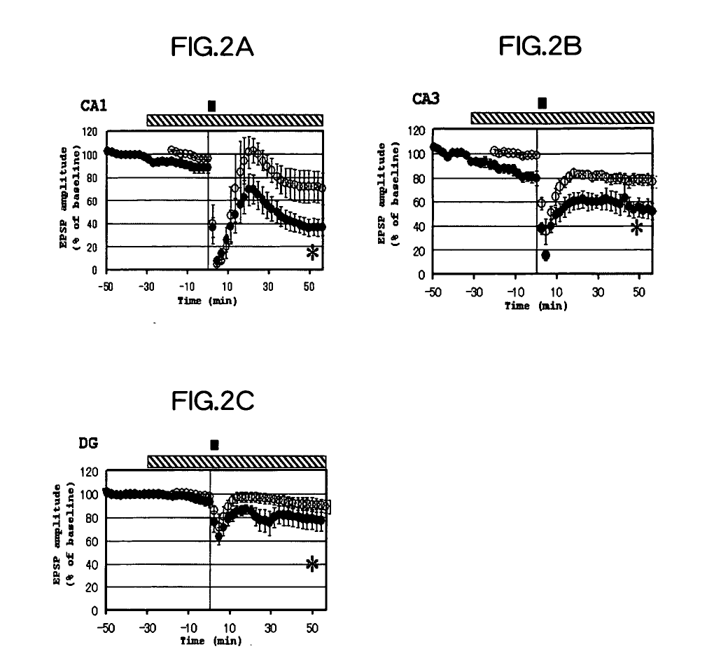

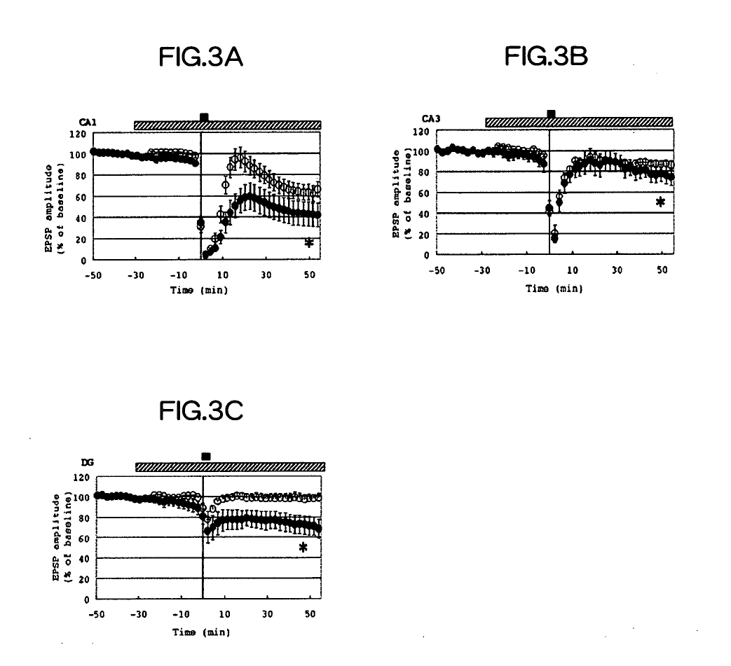

FIGS. 2 and 3 show the waves showing the result of measurement of the changes with lapse of time of the excitatory postsynaptic potential in this Example. FIG. 2 shows the effect given by estradiol on the degree of long-term depression of the excitatory postsynaptic potential induced by NMDA. In FIG. 2, an abscissa represents time (minute(s)) while an ordinate represents excitatory postsynaptic potential (%) and the bar with oblique lines shown in the upper area of each graph shows the time zone when estradiol was administered by perfusion. Further, in FIG. 2, a black dot shows the result when the first hippocampal tissue slice was brought into contact with estradiol while a white dot shows the result of a control experiment using the third hippocampal tissue slice. FIG. 2A shows the result of the experiment for cornu ammonis CA1 field, FIG. 2B shows the result of the experiment for cornu ammonis CA3 field and FIG. 2C shows the result of the experiment for dentate gyrus.

As shown in FIG. 2A, when NMDA (30 μM) was administered by perfusion for 3 minutes (for the time zone shown by a black bar) from the stage of time 0 (zero) in the control experiment (white dots) using cornu ammonis CA1 field of the third hippocampal tissue slice, a transient decrease of the excitatory postsynaptic potential reaction in the cornu ammonis CA1 field of the third hippocampal tissue slice was observed. In addition, the excitatory postsynaptic potential at the stage 50 minutes after administration of NMDA decreased to an extent of about 70% of the excitatory postsynaptic potential before administration of NMDA.

On the other hand, as shown in FIG. 2A, in the case where cornu ammonis CA1 field (black dots) of the first hippocampal tissue slice to which estradiol (1 nM) was administered by perfusion for 30 minutes (during the time zone shown by the bar with oblique lines) before administration of NMDA, the excitatory postsynaptic potential at the stage 50 minutes after administration of NMDA greatly decreased compared with that in the control experiment and, decreased to an extent of about 40% of the excitatory postsynaptic potential before administration of NMDA.

Further, as shown in FIG. 2B, even in a control experiment using cornu ammonis CA3 field using the third hippocampal tissue slice (white dots), a transient decrease in the excitatory postsynaptic potential was noted by administration of NMDA. In addition, the excitatory postsynaptic potential at the stage 50 minutes after administration of NMDA decreased to an extent of about 80% to the excitatory postsynaptic potential before administration of NMDA.

On the other hand, as shown in FIG. 2B, in the case where the cornu ammonis CA3 field (black dots) of the first hippocampal tissue slice to which estradiol (1 nM) was administered by perfusion for 30 minutes (during the time zone of the bar with oblique lines) before administration of NMDA, the excitatory postsynaptic potential at the stage 50 minutes after administration of NMDA greatly decreased compared with that in the control experiment and, decreased to an extent of about 55% of the excitatory postsynaptic potential before administration of NMDA.

Further, as shown in FIG. 2C, even in a control experiment using dentate gyrus of the third hippocampal tissue slice (white dots), a transient decrease in the excitatory postsynaptic potential was noted by administration of NMDA. In addition, the excitatory postsynaptic potential at the stage 50 minutes after administration of NMDA decreased to an extent of about 90% of the excitatory postsynaptic potential before administration of NMDA.

On the other hand, as shown in FIG. 2C, in the case where hippocampal dentate gyrus (black dots) of the first hippocampal tissue slice to which estradiol (1 nM) was administered by perfusion for 30 minutes (during the time zone of the bar with oblique lines) before administration of NMDA, the excitatory postsynaptic potential at the stage 50 minutes after administration of NMDA greatly decreased compared with that in the control experiment and, decreased to an extent of about 80% of the excitatory postsynaptic potential before administration of NMDA.

FIG. 3 shows an effect of DES on the degree of long-term depression of the excitatory postsynaptic potential induced by NMDA. In FIG. 3, each of abscissa, ordinate and black bar and white bar with oblique line on the upper area of the graph is the same as those in FIG. 2, and black dots and white dots show the result for the second hippocampal tissue slice and for the control experiment using the fourth hippocampal tissue slice, respectively. FIG. 3A shows the result for the measurement for cornu ammonis CA1 field, FIG. 3B shows the result for measurement for cornu ammonis CA3 field and FIG. 3C shows the result for measurement for dentate gyrus.

As shown in FIG. 3A, when NMDA (30 μM) was administered by perfusion for 3 minutes (for the time zone of black bar) from the stage of time 0 (zero) in the control experiment (white dots) using cornu ammonis CA1 field of the fourth hippocampal tissue slice, a transient decrease of the excitatory postsynaptic potential reaction in cornu ammonis CA1 field of the fourth hippocampal tissue slice was observed. In addition, the excitatory postsynaptic potential at the stage 50 minutes after administration of NMDA decreased to an extent of about 65% of the excitatory postsynaptic potential before administration of NMDA.

On the other hand, as shown in FIG. 3A, in the case where cornu ammonis CA1 field (black dots) of the second hippocampal tissue slice to which DES (1 nM) was administered by perfusion for 30 minutes (during the time zone of the bar with oblique lines) before administration of NMDA, the excitatory postsynaptic potential at the stage of 50 minutes after administration of NMDA greatly decreased compared with that in the control experiment and, decreased to an extent of about 40% of the excitatory postsynaptic potential before administration of NMDA.

Further, as shown in FIG. 3B, even in a control experiment using cornu ammonis CA3 field using the fourth hippocampal tissue slice (white dots), a transient decrease in the excitatory postsynaptic potential was noted by administration of NMDA. In addition, the excitatory postsynaptic potential at the stage 50 minutes after administration of NMDA decreased to an extent of about 85% of the excitatory postsynaptic potential before administration of NMDA.

On the other hand, as shown in FIG. 3B, in the case where cornu ammonis CA3 field (black dots) of the second hippocampal tissue slice to which DES (1 nM) was administered by perfusion for 30 minutes (during the time zone of the bar with oblique lines) before administration of NMDA, the excitatory postsynaptic potential at the stage 50 minutes after administration of NMDA greatly decreased compared with that in the control experiment and, decreased to an extent of about 75% of the excitatory postsynaptic potential before administration of NMDA.

Further, as shown in FIG. 3C, even in a control experiment using dentate gyrus of the fourth hippocampal tissue slice (white dots), a transient decrease in the excitatory postsynaptic potential was noted by administration of NMDA. In addition, the excitatory postsynaptic potential at the stage 50 minutes after administration of NMDA decreased to an extent of about 98% of the excitatory postsynaptic potential before administration of NMDA.

On the other hand, as shown in FIG. 3C, in the case where hippocampal dentate gyrus (black dots) of the second hippocampal tissue slice to which DES (1 nM) was administered by perfusion for 30 minutes (during the time zone of the bar with oblique lines) before administration of NMDA, the excitatory postsynaptic potential at the stage 50 minutes after administration of NMDA greatly decreased compared with that in the control experiment and, decreased to an extent of about 68% of the excitatory postsynaptic potential before administration of NMDA.

What is claimed is:

1. In a method for the evaluation of endocrine disruptive action of an object substance, a method for the evaluation of endocrine disruptive action which is characterized in comprising
a step where the object substance is brought into contact with a cell group in which an electrode is arranged,
a step where an agonist to a hormone receptor is brought into contact, as a comparative substance, with a cell group in which an electrode is arranged and
a measuring step where electrophysiological responses in the cell groups are measured via the electrode before and after contacting the object substance and before and after contacting the comparative substance, respectively.

2. In a method for the evaluation of endocrine disruptive action of an object substance, a method for the evaluation of endocrine disruptive action which is characterized in comprising
a step where the object substance is brought into contact with a cell group in which an electrode is arranged,
a step where an antagonist to a hormone receptor is brought into contact, as a comparative substance, with a cell group in which an electrode is arranged followed by bringing the object substance into contact therewith and
a measuring step where electrophysiological responses in the cell groups are measured via the electrode before and after contacting the object substance.

3. The method for the evaluation of endocrine disruptive action according to claim 1, wherein the hormone receptor is a steroid hormone receptor.

4. The method for the evaluation of endocrine disruptive action according to claim 1, wherein electrophysiological responses depending upon changes in potential of cell membrane in the cell group is measured in the measuring step.

5. The method for the evaluation of endocrine disruptive action according to claim 1, wherein the cell group is a nerve cell group.

6. The method for the evaluation of endocrine disruptive action according to claim 1, wherein the electrode contains plural electrodes regularly arranged on a substrate of a multiple-electrode probe.

7. The method for the evaluation of endocrine disruptive action according to claim 1, wherein the method further includes
a step where a stimulant causing a change in an electrophysiological response of the cell group is brought into contact with the cell group with which the object substance is contacted and the cell group with which the comparative substance is contacted respectively, and,
in the measuring step, electrophysiological responses in the cell groups before and after contacting the stimulant are further measured.

8. The method for the evaluation of endocrine disruptive action according to claim 7, wherein
the cell group is a nerve cell group and
the stimulant is a chemical substance which causes a long-term depression in the nerve cell group.

9. The method for the evaluation of endocrine disruptive action according to claim 7, wherein the contact of the stimulant with the cell group is conducted by soaking the cell group in an aqueous solution containing the stimulant.

10. The method for the evaluation of endocrine disruptive action according to claim 9, wherein the aqueous solution containing the stimulant is perfused in a predetermined container in which the cell group is placed.

11. The method for the evaluation of endocrine disruptive action according to claim 5, wherein the nerve cell group is a nerve cell group included in the brain tissue slice.

12. The method for the evaluation of endocrine disruptive action according to claim 11, wherein the brain tissue slice is a brain tissue slice of a hippocampal region.

13. The method for the evaluation of endocrine disruptive action according to claim 12, wherein the brain tissue slice is a slice which is cut vertically to a long axis of the hippocampus.

14. The method for the evaluation of endocrine disruptive action according to claim 11, wherein the brain tissue slice is a brain tissue slice having a thickness of 150 to 400 μm.

15. The method for the evaluation of endocrine disruptive action according to claim 5, wherein, in the measuring step, an excitatory postsynaptic potential of the nerve cell group is measured.

16. The method for the evaluation of endocrine disruptive action according to claim 1, wherein the agonist is a steroid hormone.

17. The method for the evaluation of endocrine disruptive action according to claim 1, wherein the agonist is estrogen.

18. The method for the evaluation of endocrine disruptive action according to claim 2, wherein the antagonist is an estrogen inhibitor.

19. The method for the evaluation of endocrine disruptive action according to claim 7, wherein the stimulant is N-methyl-D-aspartic acid.

20. The method for the evaluation of endocrine disruptive action according to claim 7, wherein 10 to 50 μM of N-methyl-D-aspartic acid is contacted as the stimulant for the cell group within a time of 10 minutes.
